(19) United States
(12) Patent Application Publication
(10) Pub. No.: US 2007/0190580 A1
(43) Pub. Date: Aug. 16, 2007

(54) Immunoassay of Fragments of Insulin-Like Growth Factor Binding Proteins

(75) Inventors: Javad Khosravi, Toronto, ON (CA); Radhakrishna G. Krishna, Friendswood, TX (US); Gopal V. Savjani, Nassau Bay, TX (US)

Correspondence Address: BECKMAN COULTER, INC., P.O. BOX 169015, MAIL CODE 32-A02, MIAMI, FL, 33116-9015, US

(73) Assignee: BECKMAN COULTER, INC., Fullerton, CA (US)

(21) Appl. No.: 11/554,190

(22) Filed: Oct. 30, 2006

Related U.S. Application Data
(60) Provisional application No. 60/731,900, filed on Oct. 31, 2005.

(51) Int. Cl. G01N 33/542

(52) U.S. Cl. 435/7.9

(57) ABSTRACT

An immunoassay of proteolytic protein fragments is described, including immunoassays of proteolytic fragments of Insulin-like Growth Factor Binding Proteins (IGFBPs). In one embodiment, a sandwich-type “two-site” immunoassay involves two different recognition antibody partners, in which one antibody is specific for a proteolytic epitope of a protein fragment, and the other is specific for the protein fragment. An assay embodiment involves a first-step capturing of the protein fragment with a specific anti-protein antibody that binds to the proteolytic epitope of the protein fragment, and a second-step detection of the bound protein fragment with an antibody directed against the protein fragment. The various embodiments of the systems and methods of the invention are exemplified by immunoassays for proteolytic fragments of Insulin-like growth factor binding proteins (IGFBPs), such as IGFBP-1, IGFBP-3, and IGFBP-5.

### CROSS REFERENCE TO RELATED APPLICATIONS

This application claims the benefit of the priority of U.S. provisional patent application No. 60/731,900, filed Oct. 31, 2005.

### BACKGROUND OF THE INVENTION

Insulin-like growth factors (IGF-I and IGF-II) belong to a family of peptides that mediate a broad spectrum of growth hormone-dependent as well as independent mitogenic and metabolic actions essential for cell growth and development (1-4). Unlike most peptide hormones, IGFs in circulation and in other physiological fluids are associated with a group of high-affinity Insulin-like growth factor binding proteins (IGFBPs) that specifically bind and modulate IGF bioactivity at the cellular level. Six structurally homologous IGFBPs with distinct molecular size, hormonal control, tissue expression and functions have been identified (4-6).

IGFBP-1, synonymous with placental protein-12 (7) and the pregnancy-associated endometrial α₁-globulin (8), is a 25-kilodalton (kDa) protein expressed and secreted by a variety of cell types, including hepatocytes, ovarian granulosa cells, and decidualized endometrium (9-11). IGFBP-1 is present in serum, is the predominant IGF binding protein in amniotic fluid, and is the major IGF binding protein in fetal and maternal circulation (9, 12-13). In both humans and animal models, elevated levels of IGFBP-1 have been found in association with fetal growth restrictions (9, 13-17).

IGFBP-1 is reportedly capable of both inhibition as well as augmentation of IGF action (4, 6). These dual functionalities of IGFBP-1 have been partly explained by posttranslational phosphorylation of amino acid residues. In the field of IGF research, a significant relationship between protein phosphorylation and immunoreactivity was first described for IGFBP-1, where as a result of differential recognition of IGFBP-1 phosphoforms by different antibodies, up to ten-fold differences in the measured concentrations of IGFBP-1 in normal adult sera were observed (20, 24). Variable recognition of IGFBP-1 phosphoforms by antibodies may result in false estimates or in inappropriate interpretations of the measured IGFBP-1 levels. Significant changes in immunoreactivity of IGFBP-5 in response to altered phosphorylation have also been observed. Immunoassays for IGFBPs have been developed where the antibody immunoreactivity is unaffected by phosphorylation of the protein (24, U.S. Pat. No. 5,747,273). Such an immunoassay allows the detection of the total concentration of the IGFBP in a sample regardless of the degree of phosphorylation.

Post-translational modification of amino acid residues, including reversible phosphorylation and proteolytic modification, is an essential and almost universal mechanism of protein activation and deactivation, and is responsible for regulating nearly all cell signaling pathways and ultimately all biological functions (50). In addition to phosphorylation of IGFBPs, posttranslational proteolysis of IGFBPs has been reported to be involved in the regulation of systemic and local IGF bioavailability. Modulation of the IGF system reportedly involves cleavage of the IGFBPs by proteases into fragments having lower affinities for IGF, leading to alterations in the IGF/IGFBP balance, and thus to increased IGF receptor activation (4, 6, 25-31). Protease modulation of IGFBP-1 has been suggested to be involved in the regulation of the IGF-dependent as well as IGF-independent actions of IGFBP-1, particularly in relation to the well established role of IGFBP-1 in fetal growth and development and in pregnancy (1-23). In this context, a number of recent publications have reported detection of IGFBP-1 proteolytic fragments in human amniotic fluid and in serum of pre-term pregnancies (32, 33) as well as in serum and urine of children with chorionic renal failure (34).

Published studies show that in normal individuals, circulating IGFBP-1 concentrations fluctuate rapidly, by 10-fold or more, in response to acute changes in insulin concentrations (9). Because of such fluctuations, IGFBP-1 levels should be measured in such individuals after a fasting period in order to obtain meaningful measurements. Fasting may not be always possible or recommended, however. The difficulty in obtaining fasting samples from pregnant women is a limitation to the assessment of IGFBP-1 in relation to fetal growth and development, and for investigations of pregnancy-related conditions. The potential masking effect of acute fluctuations in the total IGFBP-1 levels (9) on meaningful measurements would be overcome by the specific measurement of IGFBP-1 proteolytic fragment levels in relation to the total IGFBP-1 concentrations. The ability to perform such a “ratio” measurement would also be of importance in relation to pathophysiological conditions in which there is a divergence in the normal relationship between the total levels of IGFBP-1 and its circulating proteolytic fragment of interest.

Regulation of IGF-I by proteolysis of IGFBPs has been reported, including proteolysis of IGFBP-3 (4, 6, 25-31). Some reports have described production of IGFBP proteases by a variety of cell types and have associated significant enhancement of IGFBP-3 proteolysis in response to different pathophysiological conditions. Intriguing recent evidence has identified specific IGFBP proteases (29) and has indicated a positive link between IGF/IGFBP dynamics and the risk of cancer development (39-42). Cleavage of IGFBPs by proteolysis appears to be a tightly regulated mechanism (28, 29, 30), and one that would result in enhanced IGF-I dissociation from IGFBPs and increased IGF bioavailability (4, 6, 25-31). As disease associated enhancement in IGF-I activity is closely linked to its regulation by IGFBPs (43), determination of functionally active intact IGFBPs and/or their proteolytic fragments would more accurately reflect IGFBP bioactivity and, hence, the pathophysiological relevance of IGFBP circulating levels. Recent reports have described the detection of IGFBP-1 fragments in amniotic fluid and serum of pregnant females with intra-amniotic infection (32), and have reported a similar biological activity of a C-terminal fragment of IGFBP-1 to that of the intact molecule (33). The accurate quantification of IGFBPs and their various isoforms and fragments is important in view of the link between the functions of IGFBPs and diverse pathophysiological conditions, particularly in relation to human reproductive physiology (1-23). Accordingly, development of methods for quantification of the various fragments of IGFBPs would be highly valuable in the investigation of altered IGFBP proteolysis, thus aiding the better understanding of the regulation of IGFBP biological activity.

As the immunological basis of antigen-antibody interactions is largely dependent on recognition of conformational epitopes (51), the effects of protein proteolysis in relation to potential changes in immunoreactivity are of significant importance (50, 51). In traditional immunoassays using antibodies with broad specificity for target proteins, such antibodies are likely to be cross-reactive with the intact protein molecule and any proteolytic protein fragments that are present. The availability of simple immunoassays for the specific determination of proteolytic fragments of proteins, employing antibodies having specificity to the protein fragments as opposed to the intact proteins, would expedite the understanding of the regulatory mechanisms, pathophysiological roles, and clinical relevance of these proteins.

There remains a need in the art for comparatively simple and reliable quantitative immunoassay methodologies and compositions that permit the measurement of proteolytic protein fragments and provide for large scale sample analysis by manual and/or fully automated operation.

### SUMMARY OF THE INVENTION

Methods and systems relating to immunoassays for the quantification of protein fragments are described herein. More specifically, the immunoassays described herein relate to the quantification of proteolytic fragments of proteins, including Insulin-like growth factor binding proteins (IGFBPs).

Various embodiments of methods and systems described herein provide the ability to quantify proteolytic fragments of proteins, such as fragments of IGFBPs, using the advantages and simplicity of the conventional immunoassay format. Providing an antibody with specificity for a proteolytic epitope of a protein fragment to allow the specific quantification of such protein fragments represents a novel approach to immunoassay of proteolytic protein fragments, as exemplified here for IGFBP-1.

Embodiments described herein will expedite investigations of protein proteolysis, including proteolysis of IGFBPs such as IGFBP-1, and are useful to correct or minimize the effect of significant daily fluctuations in circulating total IGFBP-1 levels (9) on measurements of IGFBP-1 that would potentially obscure detection of changes in the levels of IGFBP-1 fragments. Such embodiments are valuable in pregnancy investigations, where fasting samples are difficult to obtain.

Embodiments herein disclose an immunoassay system comprising a first antibody and a second antibody, wherein the first antibody binds to a proteolytic epitope of a protein fragment, and wherein the second antibody binds to the protein fragment.

An additional embodiment discloses an immunoassay method for measuring an amount of a protein fragment in a sample, comprising the steps of binding a first antibody to a proteolytic epitope of a protein fragment in a sample, thereby creating a bound first antibody; binding a second antibody to the protein fragment, thereby creating a bound second antibody; measuring an amount of the bound second antibody; and measuring an amount of the protein fragment in the sample based on the amount of the bound second antibody.

Other possible embodiments disclose an immunoassay kit for measuring an amount of a protein fragment in a sample, comprising a first antibody and a second antibody, wherein the first antibody binds to a proteolytic epitope in a protein fragment and the second antibody binds to the protein fragment; a solid support coupled with the first antibody; and a label coupled with the second antibody.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the various embodiments of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 is a graph showing the concentration (ng/mL) of IGFBP-1 C-domain peptide 146-259 (SEQ ID NO: 3) measured by enzyme-linked immunosorbent assay (ELISA), absorbance at 450 nm. IGFBP-1 fragment 146-259 was dissolved appropriately in a standard Tris-based matrix buffer at the indicated concentrations. Values shown are the means of duplicate measurements. See Example 1.

FIG. 2A is a graph showing the relationship between IGFBP-1 C-terminal and total IGFBP-1 (ng/mL) immunoreactivity measured in serum samples from random adults. Values are the mean of duplicate measurements. The slope calculation formulae are y=0.139x+0.087; r²=0.96.

FIG. 2B is a graph showing the relationship between IGFBP-1 C-terminal and total IGFBP-1 (ng/mL) immunoreactivity measured in serum samples from first and second trimester pregnancies. Values are the mean of duplicate measurements. The slope calculation formulae are y=0.128x+1.93; r²=0.75.

FIG. 3 shows the distribution of IGFBP-1 C-terminal proteolytic fragment and total IGFBP-1 concentration ratios: Box plots show the distributions of IGFBP-1 C-terminal proteolytic fragment/Total IGFBP-1 levels in (1) pregnancy samples and (2) in random adult samples.

### DETAILED DESCRIPTION OF THE INVENTION

I. Systems of the Invention

An immunoassay system, as described herein, comprises a first antibody and a second antibody. The first antibody binds to a proteolytic epitope of a protein fragment. The second antibody binds to the protein fragment. In one embodiment, the composition is an intermediate provided by the first antibody bound to the “target” protein, which is bound to the second antibody. As described in more detail below, the first antibody is optionally bound to a solid support. In another embodiment, the second antibody is optionally bound to a label. In another embodiment, the first antibody is bound to the target protein at a first proteolytic epitope, and the second antibody binds to the target protein at a second epitope. The binding of the first antibody to the first epitope does not interfere with the binding of the second antibody to the second epitope. In another embodiment, the first proteolytic epitope differs from the second epitope.

In various embodiments, the proteolytic epitope recognized by the first antibody is an epitope which is present in a protein fragment as a result of proteolytic cleavage of the protein. Such an epitope results from the exposure of amino acid residues on the surface of a protein fragment after the protein is cleaved by a protease. Thus, a proteolytic protein fragment contains an epitope(s) that is different from epitopes available for antibody binding to the intact protein. In one embodiment, the amino acid residues in the proteolytic epitope are located within a primary amino acid sequence region of the proteolytic fragment of the protein which is in proximity to the amino acid sequence site that is cleaved by the protease. In various embodiments, antibodies are generated against such a proteolytic epitope for use in immunoassay systems to bind to a protein fragment containing such an epitope. Such immunoassay systems are useful for the specific measurement of a proteolytic fragment of a protein in a sample that also contains the intact protein or different protein fragments.

Another embodiment of a system of this invention is a product or collection of the individual components that make up the system. An embodiment of such a system is an immunoassay kit for measuring an amount of a protein fragment in a sample. In one embodiment, such a kit comprises a first antibody and a second antibody, wherein the first antibody binds to a proteolytic epitope of a protein fragment, and the second antibody binds to the protein fragment. According to such an embodiment, the kit also contains a solid support coupled with the first antibody and a label coupled with the second antibody. Suitable examples of solid supports are identified below. Suitable examples of labels for use in the kit are similarly identified below. The kit also contains optional additional components for performing assay methods described herein. Such optional components are independently selected from containers, mixers, instructions for assay performance, labels, supports, and reagents necessary to couple the antibody to the support or label.

Descriptions of the components of these compositions, products and kits including the antibodies, target protein, supports, labels and optional kit components are provided in more detail below.

II. Methods of the Invention

An embodiment of a method of the invention is an immunoassay method for measuring an amount of a protein fragment in a sample, comprising the steps of binding a first antibody to a proteolytic epitope of the protein fragment, thereby creating a bound first antibody; binding a second antibody to the protein fragment, thereby creating a bound second antibody; measuring an amount of the bound second antibody; and measuring the amount of the protein fragment in the sample based on the amount of bound second antibody.

In one embodiment, a one-step assay (simultaneous incubation of sample plus detection antibody) is useful. In another embodiment, a two-step assay (sequential incubation of sample and the detection antibody) is useful. A two-step assay is preferable in the case where other protein molecules could compete for binding to the detection antibody.

In an embodiment of an immunoassay referred to as immunometric, “two-site” or “sandwich” immunoassay, the analyte is bound to or sandwiched between two antibodies that bind to different epitopes on the analyte. Representative examples of such immunoassays include enzyme immunoassays or enzyme-linked immunosorbent assays (EIA or ELISA), immunoradiometric assays (IRMA), fluorescent immunoassays, lateral flow assays, diffusion immunoassays, immunoprecipitation assays, and magnetic separation assays (MSA). In one such assay, a first antibody, which is described as the “capture” antibody, is bound to a solid support, such as a protein coupling or protein binding surface, colloidal metal particles, iron oxide particles, or polymeric beads. One example of a polymeric bead is a latex particle. In such an embodiment, the capture antibody is bound to or coated on a solid support using procedures known in the art. Alternatively, the capture antibody is coupled with a ligand that is recognized by an additional antibody that is bound to or coated on a solid support. Binding of the capture antibody to the additional antibody via the ligand then indirectly immobilizes the capture antibody on the solid support. An example of such a ligand is fluorescein.

The second antibody, which is described as the “detection” antibody, is coupled or conjugated with a label using procedures known in the art. Examples of suitable labels for this purpose include a chemiluminescent agent, a colorimetric agent, an energy transfer agent, an enzyme, a substrate of an enzymatic reaction, a fluorescent agent and a radioisotope. In one embodiment, the label includes a first protein such as biotin coupled with the second antibody, and a second protein such as streptavidin that is coupled with an enzyme. The second protein binds to the first protein. The enzyme produces a detectable signal when provided with substrate(s), so that the amount of signal measured corresponds to the amount of second antibody that is bound to the analyte. Examples of enzymes include, without limitation, alkaline phosphatase, amylase, luciferase, catalase, beta-galactosidase, glucose oxidase, glucose-6-phosphate dehydrogenase, hexokinase, horseradish peroxidase, lactamase, urease and malate dehydrogenase. Suitable substrates include, without limitation, TMB (3,3′,5,5′-tetramethyl benzidine, OPD (o-phenylene diamine), and ABTS (2,2′-azino-bis(3-ethylbenzthiazoline-6-sulfonic acid).

An additional embodiment of an immunoassay method is designed for measuring an amount of a protein fragment relative to a total amount of the protein in a sample. Such a method comprises the steps of contacting a first antibody with a target protein in a sample, where the target protein comprises a proteolytic epitope. The first antibody binds to the proteolytic epitope, thereby creating a bound first antibody. A second antibody is contacted with the sample, and binds to the protein fragment, thereby creating a bound second antibody. The amount of bound second antibody is measured. The amount of the protein fragment in the sample is measured based on the amount of bound second antibody. The amount of total target protein in the sample is determined, and related to the amount of the protein fragment in the sample. One possible embodiment involves calculating the concentrations of the protein fragment and the total proteins using the amounts of these proteins measured in a sample. In another embodiment, relating the amount of the protein fragment to the amount of total protein in the sample involves calculating a ratio of the amount of the protein fragment and the amount of total protein in the sample.

The various embodiments of the described systems and methods are used to measure proteolytic variants or fragments of a protein. In embodiments herein, the first antibody that binds to a protein fragment binds to a first proteolytic epitope in the protein fragment, and the second antibody binds to a second epitope in the protein fragment. In one embodiment, the first epitope is different from the second epitope, so that binding of the first antibody to the protein fragment does not interfere with the binding of the second antibody to the protein fragment. The compositions and methods described herein provide an immunoassay approach for the specific quantification of proteolytic protein fragments that is applicable to both manual and automated immunoassay platforms. The development and performance characteristics of novel enzyme-linked immunosorbent assays (ELISAs) for specific quantification of proteolytic fragments of IGFBP proteins are described. The features of the assays described herein have been demonstrated by using IGFBP-1 as the target protein with a C-terminal fragment of IGFBP-1 generated by proteolytic cleavage of IGFBP-1 between amino acid residues 145 and 146. Thus, the compositions and methods described in the examples below involve an anti-IGFBP-1 antibody that binds to a proteolytic epitope in the C-terminal fragment of IGFBP-1, in combination with an antibody that binds to the C-terminal IGFBP-1 fragment.

Additional embodiments disclose immunoassay methods for diagnosing a condition related to a proteolytic fragment of an IGFBP in an individual, comprising the steps of obtaining a body fluid from an individual; measuring an amount of a proteolytic fragment of an IGFBP in the body fluid using immunoassay systems described herein; and comparing the amount of the IGFBP proteolytic fragment in the body fluid to a reference level of the IGFBP proteolytic fragment in healthy individuals without the condition, wherein an elevated amount of the proteolytic fragment of an IGFBP above the reference level indicates the individual has the condition. Other embodiments disclose immunoassay methods for diagnosing intra-amniotic infection in a pregnant individual, comprising the steps of obtaining a body fluid from a pregnant individual; measuring an amount of a proteolytic fragment of IGFBP-1 in the body fluid according to immunoassay methods described herein; and comparing the amount of the proteolytic fragment of IGFBP-1 in the body fluid to a reference level of the proteolytic fragment of IGFBP-1 in healthy pregnant individuals without intra-amniotic infection, wherein an elevated amount of the proteolytic fragment of IGFBP-1 in the body fluid above the reference level indicates intra-amniotic infection in the pregnant individual.

III. Components of the Systems and Methods of the Invention

A. Target Proteins and Samples

An example of a target protein with proteolytic protein fragment that is measured using embodiments of the present invention is an IGFBP. Examples of IGFBPs are IGFBP-1, IGFBP-3, and IGFBP-5. Other proteins that have proteolytic protein fragments or variants and that are suitable for analysis by the methods described herein may be readily selected from among proteins known in the art, including a variety of enzymes, growth factors and transcription factors, among others.

Certain target proteins are present in ternary protein complexes (36), and as such are less accessible for binding by antibodies to their epitopes. Such target proteins are also able to be measured using the compositions and methods described herein. However, such targets are subject to additional method steps to permit changes in the protein structure to permit binding by the antibodies, e.g., inducing a change in the ternary structure. Such changes include, without limitation, conventional treatment to permit exposure of the epitopes for binding by the first and/or second antibodies.

In various embodiments, a sample in which a proteolytic protein fragment is measured is a biological fluid in which the protein naturally occurs. An example of a useful biological fluid that contains protein fragments includes a serum sample, such as a human serum sample. Examples of human serum samples include non-pregnant serum, pregnancy serum from the first, second or third trimester. Still another suitable biological sample is amniotic fluid. Still other biological fluids that contain protein fragments suitable for the assays described herein may be selected from among known fluids, including without limitation, whole blood, plasma, urine, saliva, tears, cerebrospinal fluid, among others. Other samples may include non-naturally occurring or synthetic fluids or solutions containing fragments of proteins.

B. Antibodies

Antibodies useful in the various embodiments of the systems and methods described herein include commercially available antibodies and antibody fragments, as well as any novel antibodies generated to bind a suitable epitope on the designated target protein. The antibodies used in various embodiments exemplified herein are monoclonal or polyclonal in nature. Other antibodies and antibody fragments, such as recombinant antibodies, chimeric antibodies, humanized antibodies, antibody fragments such as Fab or Fv fragments, as well as fragments selected by screening phage display libraries, and the like are also useful in the compositions and methods described herein.

Methods for preparation of monoclonal as well as polyclonal antibodies are now well established (Harlow E. et al., 1988. Antibodies. New York: Cold Spring Harbour Laboratory). In one embodiment, antibodies are raised against recombinant human IGFBPs, synthetic fragments thereof, or IGFBP/IGF protein complexes, such as may be purified from human sera. Polyclonal antibodies are raised in various species including but not limited to mouse, rat, rabbit, goat, sheep, donkey and horse, using standard immunization and bleeding procedures. Animal bleeds with high titres are fractionated by routine selective salt-out procedures, such as precipitation with ammonium sulfate and specific immunoglobulin fractions being separated by successive affinity chromatography on Protein-A-Sepharose and leptin-Sepharose columns, according to standard methods. The purified polyclonal as well as monoclonal antibodies are then characterised for specificity and lack of cross-reactivity with related molecules. Such characterization is performed by standard methods using proteins, for example IGFBPs, labeled with a tracer such as a radioisotope or biotin in competition with increasing levels of unlabeled potential cross-reactants for antibody binding. In some embodiments, further purification is required to obtain highly specific antibody fractions or for selection of higher affinity antibody fractions from a polyclonal pool. In the case of monoclonal antibodies, care is taken to select antibodies with good binding characteristics and specificity not only for the immunogen, but also for the native circulating molecules, particularly when a recombinant molecule or peptide antigen is used for immunization. Cross-reactivity studies are further evaluated by other standard methods such as the well-established sodium dodecyl sulphate-polyacrylamide gel electrophoresis (SDS-PAGE) and Western immunoblot methods under reducing and non-reducing conditions. Evaluation of protein immunoreactivity detected in serum samples fractionated by high performance liquid chromatography (HPLC) is also used to roughly define the molecular weight profile of the protein detected (32, 37).

Monoclonal antibodies are prepared according to well established standard laboratory procedures (“Practice and Theory of Enzyme Immunoassays” by P. Tijssen (In Laboratory Techniques in Biochemistry and Molecular Biology, Eds: R. H. Burdon and P. H. van Kinppenberg; Elsevier Publishers Biomedical Division, 1985)), which are based on the original technique of Kohler and Milstein (Kohler G., Milstein C. Nature 256:495, 1975). This technique is performed by removing spleen cells from immunized animals and immortalizing the antibody producing cells by fusion with myeloma cells or by Epstein-Barr virus transformation, and then screening for clones expressing the desired antibody, although other techniques known in the art are also used. Antibodies are also produced by other approaches known to those skilled in the art, including but not limited to immunization with specific DNA.

For use in the immunoassays described herein, antibodies are purified using standard antibody purification schemes. In various embodiments, both monoclonal and polyclonal antibodies are purified by affinity chromatography over Protein-A columns. Alternatively, the antibodies are purified by affinity chromatography over a gel column containing immobilized antigen protein using standard methods.

Another consideration for selection of the appropriate antibody for use in the systems and methods described herein is the ability of the capture antibody and the detection antibody to bind simultaneously to a given protein molecule. In one embodiment involving an IGFBP, the anti-IGFBP binding site of the capture antibody (proteolytic epitope) is different from the epitope to which the detection antibody binds, thus allowing for simultaneous binding of the capture and detection antibodies and detection of the proteolytic fragments of the protein. In the case of significant overlap of epitopes and a resulting poor binding response, it is within the skill of one in the art to select a different anti-IGFBP antibody as the capture or detection antibody. In some embodiments an antibody binding site is not entirely available on the surface of the protein, for example where the protein is mainly present in the sample in a complex with one or more other proteins, and is less accessible for binding to the capture or detection antibodies. In such a circumstance, techniques known in the art are used to expose the antibody binding sites, such as partial protein denaturation or buffer modification.

As known in the art, the capture antibody is coupled with or linked to various solid phase supports using standard non-covalent or covalent binding methods, depending on the required analytical and/or solid-phase separation requirements. The solid-support is in the form of test tubes, beads, microparticles, filter paper, membranes, glass filters, magnetic particles, glass or silicon chips or other materials and approaches known to those skilled in the art. The use of microparticles, particularly magnetizable particles, that have been directly coated with the antibody (magnetic particles-capture antibody) or particles that have been labelled with a universal binder (e.g., avidin or anti-species antibody) is useful for significantly shortening the assay incubation time. These along with other alternative approaches known in the art allow for assay completion within minutes without limiting the required sensitivity. The use of magnetizable particles or similar approaches allow for convenient automation of the technology on the widely available immunoanalyzers.

The detection antibody used for detection of the protein fragment is either directly coupled with a reporter molecule, or detected indirectly by a secondary detection system. The latter is based on several different principles known in the art, including antibody recognition by a labelled anti-species antibody and other forms of immunological or non-immunological bridging and signal amplification detection systems (e.g., the biotin-streptavidin technology). The signal amplification approach is used to significantly increase the assay sensitivity and low level reproducibility and performance. The label used for direct or indirect antibody coupling is any detectable reporter molecule. Examples of suitable labels are those widely used in the field of immunological and non-immunological detection systems, such as fluorophores, luminescent labels, metal complexes and radioactive labels, as well as moieties that could be detected by other suitable reagents such as enzymes, or various combinations of direct or indirect labels such as enzymes with luminogenic substrates.

C. Buffers

The standard immunoassay matrix is a buffer-based solution containing a carrier protein (e.g., 0.05 mol/L Tris, pH 7.4, 9g/L NaCl, 5g/L BSA, 0.1 g/L Proclin 300) or a human or animal serum including but not limited to normal goat serum (NGS), normal equine serum (NES), or new born calf serum (NBCS). Other standard matrix preparations known in the art are also useful. One of skill in the art may readily select a buffer for various embodiments, such as those based on Tris, Borate, Phosphate or Carbonate capable of broad pH ranges.

IV Embodiments of the Methods and Systems of the Invention

Various embodiments of systems and methods to measure proteolytic fragments of IGFBPs using an immunoassay approach are described herein. The immunoassay is based on a design in which the IGFBP fragment is captured by an anti-IGFBP antibody that binds to a proteolytic epitope of the IGFBP fragment, and detected by a second antibody against the IGFBP fragment.

In various embodiments of the methods of the invention, any sample and antibody volumes and incubation times are within the skill of one in the art to alter. These methods and systems include common modifications used in conventional immunoassays, and any modification known to those skilled in the art. In various embodiments, the assay design is homogeneous or heterogeneous, depending on the particular application of the assay and the need for speed, sensitivity, accuracy and convenience.

Various embodiments allow the accurate tracking of changes in the state of protein proteolysis in response to changes in pathophysiological conditions of interest. Availability of such immunoassays and methods for their use will facilitate investigations of the pathophysiological roles and potential diagnostic values of proteolysis of proteins. The specific quantification and monitoring of changes in the level of protein proteolysis are more informative in relation to measuring the total protein immunoreactivity than the currently available immunoassays, for example, for IGFBP-1 (32) or IGFBP-3 (37). Relating the amounts or concentrations of proteolytic protein fragments to the total amounts or concentrations of the protein, such as by using a “ratio” determination, is useful when assessing pathophysiological conditions in which changes in the proteolysis levels of the protein are greater than changes in its total immunoreactivity levels, such as in cases where the levels of IGFBP-1 fragments are altered independently of the total IGFBP-1 levels. Such a ratio measurement is useful for correcting significant fluctuations in the total IGFBP-1 levels that occur in response to acute changes in insulin levels (9) and for indicating conditions associated with a pathophysiological divergence in the IGFBP-1 C-terminal proteolytic fragment versus total IGFBP-1 levels. Such an approach is also be useful for monitoring relative changes in pathophysiological levels of other proteolytic isoforms of IGFBPs, including but not limited to other proteolytic fragments of IGFBP-1.

The following examples are given for the purpose of illustrating various embodiments of the invention and are not meant to limit the present invention in any fashion:

### EXAMPLE 1

### IGFBP Fragment Immunoassay

Sample Preparation.

Serum samples from non-pregnant females (n=29, age 17-48, median), and from first (n=38, age, median) and second (n=29) trimester pregnancies were obtained from Lenetix Medical Screening Laboratory Inc., (New York, N.Y.). These specimens were residuals from routine or research test samples. After collection, blood samples were allowed to clot and were then separated. After clinical testing, the residuals were stored at −20° C. and used for the present studies within 3 months. Amniotic fluids (n=20) from second trimester pregnancies (15-18 week gestations) and samples from pretoneal cavity fluids (n =24) were obtained from clinical laboratories in Toronto, ON, Canada. The samples were residuals from routine clinical test samples and were stored at −70° C. for fewer than 4 months before use.

Reagents

Horseradish peroxidase (HRP) was obtained from Scripps Labs., San Diego, Calif. The Tetramethylbenzidine (TMB) microwell peroxidase substrate system was from Neogen Corporation, Lexington, Ky. Sulfosuccinimidyl 4-(N-maleimidomethyl)cyclohexane-1-carboxyl-ate (sulfo-SMCC) and 2-iminothiolane were purchased from Pierce, Rockford, Ill. Enzyme immunoassay grade alkaline phosphatase (ALP) was obtained from Boehringer Mannheim, Indianapolis, Ind. All other chemical reagents were of the highest quality and were obtained from Sigma Chemical, St. Louis, Mo. or Amresco, Solon, Ohio. Microtitration strips and frames were products of Greiner International, Germany.

Human IGFBP-1, purified from human amniotic fluid according to previously described methods (35), and synthetic IGFBP-1 peptides were obtained from Diagnostic Systems Laboratories, Inc. (DSL, Webster, Tex.). The intact IGFBP-1 preparation was calibrated against pure recombinant human IGFBP-1.

Antibodies.

Mouse monoclonal and affinity purified goat polyclonal antibodies against the intact IGFBP-1 and its various synthetic fragments were obtained from DSL (Webster, Tex.). The specificity of the antibodies for intact IGFBP-1 has been described previously (24, U.S. Pat. No. 5,747,273). Among these antibodies, a monoclonal anti-intact IGFBP-1 antibody that was unaffected by the state of IGFBP-1 phosphorylation or IGFBP-1 binding to IGF-I was previously used in designing an immunoassay for Total IGFBP-1 (24, U.S. Pat. No. 5,747,273).

Among the candidate antibodies against IGFBP-1 and its fragments, nine different monoclonal and polyclonal antibodies raised against the various IGFBP-1 peptides, and also intact IGFBP-1, were selected for evaluation. The initial evaluation involved pair-wise assessment of all possible antibody combinations for detecting the various IGFBP-1 peptides, particularly the C-domain (C-terminal) IGFBP-1 peptide (146-259, SEQ ID NO. 3), IGFBP-1 peptide (151-259, SEQ ID NO. 4), IGFBP-1 peptide (171-259, SEQ ID NO. 5) and the intact (native) IGFBP-1. Antibody combinations affording preferential binding to the IGFBP-1 C-terminal peptides relative to their responses to the intact IGFBP-1 molecule were evaluated in more detail.

Synthetic Peptides.

Development of an IGFBP-1 fragment immunoassay as described herein was based on a systematic approach to the design and synthesis of IGFBP-1 fragment peptides for immunization, as well the screening, selection, and purification of the candidate polyclonal or monoclonal antibodies. In an approach to the immunoassay of IGFBP-1 fragments, various synthetic IGFBP-1 fragments were needed for standard preparation and determination of assay performance and comparative cross-reactivity versus intact (native) IGFBP-1.

Production and purification of the synthetic IGFBP-1 peptides was based on well-established approaches to peptide synthesis and purification known to those skilled in the art. The general approach involved designing a number of peptides representing overlapping amino acid sequences within the C-terminal proteolytic fragment of IGFBP-1 and empirical selection of candidate peptides for immunization. The peptides were synthesized for the initial feasibility studies and subsequent assay development.

The synthetic peptides evaluated herein included IGFBP-1 peptides encompassing amino acid sequences of (146-177, SEQ ID NO: 1); (151-182, SEQ ID NO. 2), (146-259, SEQ ID NO. 3), (151-259, SEQ ID NO. 4), and (171-259, SEQ ID NO. 5).

Synthetic peptides designed for generating antibodies for the measurement of IGFBP-1 C-terminal proteolytic fragments herein include:

| | | |
|---|---|---|
| SEQ ID NO. 1: | | |
| IGFBP-1: | 146-177: | EDHSILWDAISTYDGSKALHVTNIKKWKE |
| | | PCR |
| SEQ ID NO. 2: | | |
| IGFBP-1: | 151-182: | LWDAISTYDGSKALHVTNIKKWKEPCRIE |
| | | LYR |

Recombinant fragments of IGFBP-1 designed for calibrations and controls in two-site immunoassays described herein include:

| | | |
|---|---|---|
| SEQ ID NO. 3: | | |
| IGFBP-1: | 146-259: | MEDHSILWDAISTYDGSKALHVTNIKKWK |
| | | EPCRIELYRVVESLAKAQETSGEEISKFL |
| | | YPNCNKNGFYHSRQCETSMDGEAGLCWCV |
| | | YPWNGKRIPGSPEIRGDPNCQIYFNVQN |
| SEQ ID NO. 4: | | |
| IGFBP-1: | 151-259: | MLWDAISTYDGSKALHVTNIKKWKEPCRI |
| | | ELYRVVESLAKAQETSGEEISKFLYPNCN |
| | | KNGFYHSRQCETSMDGEAGLCWCVYPWNG |
| | | KRIPGSPEIRGDPNCQIYFNVQN |
| SEQ ID NO. 5: | | |
| IGFBP-1: | 171-259: | MKWKEPCRIELYRVVESLAKAQETSGEEI |
| | | SKFLYPNCNKNGFYHSRQCETSMDGEAGL |
| | | CWCVYPWNGKRIPGSPEIRGDPNCQIYFN |
| | | VQN |

Peptides were synthesized on solid phase using FMOC chemistry (44) and purified to 90% purity on reverse phase high pressure liquid chromatography with C-18 columns using trifluoroacetic acid and acetonitrile system (45, 46). Purified synthetic peptides were conjugated with carrier proteins and polyclonal antisera were generated, and screened with corresponding I-125 labeled peptides. Antibodies were purified from assay-qualified antisera by affinity ligand chromatography. Monoclonal antibodies were prepared using standard hybridoma technology, and the ascetic fluids were purified on protein-A affinity columns (47). Recombinant IGFBP-1 fragment sequences were designed basing on the proteolytic cleavage site of IGFBP-1 between amino acids 145-146, and were prepared as non-phosphorylated polypeptides in E. coli according to standard methods (48).

Reagent Preparation Protocols.

Antibody was coated to microwells (250-1000 ng/100 μL/well) according to protocols previously described (36-38). Antibody conjugation to biotin or HRP was conducted as previously described (36-38). Standards were prepared by appropriately diluting the various IGFBP-1 peptides into various standard matrix buffers to produce the desired standard concentrations in arbitrary units. The various standard preparations were initially assayed for their response in the candidate IGFBP-1 Peptide ELISAs, and the fragment with the highest relative response was selected for ELISA calibration. Cross-reactivities of the candidate IGFBP-1 Peptide ELISAs with the remaining peptides and intact IGFBP-1 were compared as percentages of the assay response to the selected peptide used for calibration. Among the various peptides, the highest ELISA binding signal was generated in response to IGFBP-1 peptide 146-259, which was, thus, used for calibration of the assay. For the ELISAs, a Tris-based assay buffer (0.05 mol/L Tris, pH 7.4, 9g/L NaCl, 5g/L BSA, 0.1 g/L Proclin 300) or a sodium phosphate-based assay buffer (0.05 M NaPO4, pH 7.4, containing 0.2.5 g BSA, 9 g NaCl, 0.5 mL twen-20, 50 mL NGS, and 2.5 mL procline 300 per litre) was selected.

The anti-IGFBP antibodies were purified using standard antibody purification schemes. Both monoclonal and polyclonal antibodies were purified by affinity chromatography over Protein-A columns or by affinity chromatography over a gel column containing immobilized IGFBP using standard methods.

Assay Protocols.

In one embodiment, a one-step assay (simultaneous incubation of sample plus detection antibody) was performed; in another embodiment, a two-step assay (sequential incubation of sample and the detection antibody) was performed.

IGFBP antibody evaluations in pair-wise combinations were conducted using conventional methods. Conditions affording a reasonable response were selected and evaluated further.

The IGFBP-1 C-terminal peptide fragment ELISAs involved the steps of addition of standards, samples or controls (0.025 mL) and the assay buffer (0.10 mL) in duplicate to the anti-IGFBP-1 peptide antibody pre-coated wells, followed by a one-hour incubation at room temperature with continuous shaking. The wells were then washed five times (×5) and incubated for one hour as above with 0.10 mL/well of the detection anti-IGFBP-1 peptide antibody. After an additional washing step, the wells were incubated with 0.1 mL/well TMB/H₂O₂ substrate solution and incubated for an additional 10 minutes, as above. Stopping solution (0.1 mL) was then added and absorbance was measured by dual wavelength measurement at 450 nm with background wavelength correction set at 620 nm. ELISA absorbance measurements were performed with the Labsystems Multiskan Multisoft microplate reader (Labsystems, Helsinki, Finland). The composition of the coating and blocking buffers and the antibody coating procedure to microtitration wells as well as the wash and stopping solutions were as described previously (24, 37).

Coupling of the detection antibodies to HRP was performed as described (24, 37). The coupling reaction involved activation of the enzyme with sulfo-SMCC and its subsequent conjugation to the detection antibody, which had been activated by 2-iminothiolane. The stock HRP-conjugated antibody solution was diluted at least 1000-fold prior to use.

Standards were IGFBP-1 C-domain peptide 146-259 (SEQ ID NO. 3) diluted in the standard matrix to give standard values of 0, 2.5, 5, 10, 25, and 50 ng of IGFBP-1 peptide per mL. The standards were stable for at least 4 days at 4° C. The quality control samples used were fresh serum samples containing various levels of immunoreactivity.

IGFBP-1 Fragment ELISA Validation Procedures.

The lower limit of detection (sensitivity) was determined by interpolating the mean plus two standard deviations (2SD) of 12 replicate measurements of the zero calibrator (NBCS). The intra-assay coefficients of variability (CVs) were determined by replicate analysis (n=12) of five samples at different concentrations in one run and inter-assay CVs by duplicate measurement of samples in 12 separate assays. Recovery was assessed by adding 25 μL of IGFBP-1 peptide 146-259 to 225 μL of four different samples with low levels of endogenous immunoreactivity, and analyzing the supplemented and un-supplemented samples. Percent recovery was determined by comparing the amount of added IGFBP-1 peptide with the amount measured after subtracting the endogenous concentration. Linearity was tested by analyzing three serum samples serially diluted (2- to 8-fold) in the zero calibrator of the assay.

Other Assays.

Total IGFBP-1 and non-phosphorylated IGFBP-1 were assayed as previously reported (32), using Diagnostic Systems Laboratories, Inc. (Webster, Tex.) Total and Non-phosphorylated IGFBP-1 ELISA immunoassays.

Data Analysis.

ELISA data were analyzed using the data reduction software package included with the instrumentation, using cubic spline (smoothed) curve fit. Descriptive data are presented as the mean, median, and standard deviation unless otherwise specified. Linear regression analysis was performed by the least-squares method, and correlation coefficients were determined by the Pearson method. The plotting and statistical analysis were performed using SigmaPlot and SigmaStat software (Systat Software Inc, Point Richmond, Calif. 94804-2028).

IGFBP-1 C-Terminal Peptide Fragment ELISA.

Assessment of panels of well characterized IGFBP-1 C-terminal proteolytic fragment peptides in combination with panels of polyclonal or monoclonal antibodies provided information on antibody binding characteristics, particularly identifying antibody combinations capable of specific detection of the C-terminal proteolytic fragment of IGFBP-1. Combinations of a polyclonal capture antibody against IGFBP-1 peptide (151-182, SEQ ID NO. 2) and a polyclonal detection antibody against IGFBP-1 peptide (146-177, SEQ ID NO. 1) demonstrated high preferential specificity for IGFBP-1 C-domain peptide (146-259, SEQ ID NO. 3) and to a lesser extent to IGFBP-1 C-domain peptide (151-259, SEQ ID NO. 4). The binding to IGFBP-1 C-domain peptide (171-259, SEQ ID NO. 5) was comparatively low to undetectable. The preferential specificity of the assay for IGFBP-1 fragments was further demonstrated by its relatively low cross-reactivity with intact (native) IGFBP-1. Collectively, the data shows preferential assay specificity for epitopes within linear sequences in the N-terminal region of the above peptide fragments, which are located within the region of proteolytic cleavage of the intact IGFBP-1 molecule, between amino acid residues 145-146. Such specificity is supported by the finding that the recognized determinant is apparently lost in the N-terminally shorter IGFBP-1 171-259 fragment (SEQ ID NO. 5), and is relatively inaccessible or modified within the ternary structure (conformation) of the native IGFBP-1 molecule. The observed binding characteristics also show that the antibodies are useful for detecting N-terminal proteolytic fragments as well as C-terminal proteolytic fragments of IGFBP-1, provided that the fragments include the favorable binding sequences (e.g., IGFBP-1 N-terminal sequences extending, for example, to amino acid residue 171).

As per conventional immunoassays, pair-wise antibody selection was based on the relative binding responses in relation to non-specific binding signal (NSB) generated by the zero-dose standard signal-to-noise ratios). Useful analytical performance characteristics were obtained with a coating antibody concentration of 5 mg/L (500 ng/0. 1 mL per well), a detection antibody concentration of 0.1-0.25 mg/L (10-25 ng/0.1 mL per well), a sample size of 0.025 mL, a first- and second-step room temperature incubation of 1hr and 1hr, respectively, and a 10-min substrate development step.

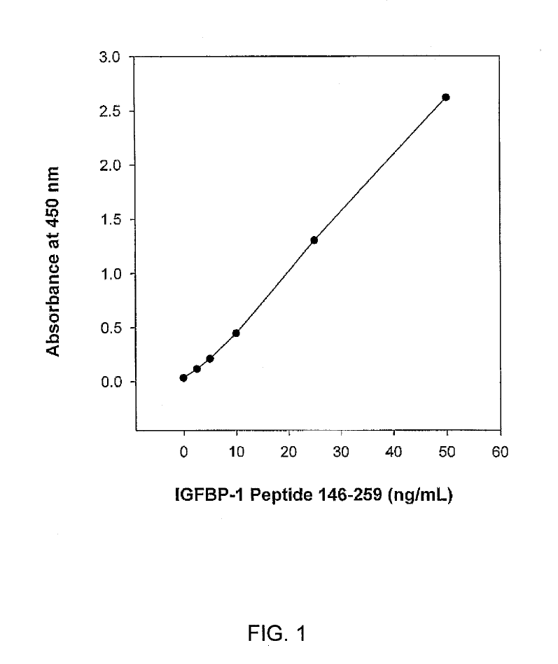

Standard curve results for the IGFBP-1 C-terminal fragment ELISA are shown in FIG. 1. As a representative example, the analytical performance characteristics of the IGFBP-1 C-terminal fragment ELISA were assessed in more detail using conventional approaches. The relevant performance data are summarized in Table 1.

TABLE 1
C-Terminal Fragment IGFBP-1 ELISA: Immunoassay Validation Data
Assay parameter

| | |
|---|---|
| Detection limit (ng/ml) | 0.20 |
| Standard range (ng/ml) | 2.5-50 |
| Intra-assay CV, % | 1.3-5.1 |
| Inter-assay CV, % | 4.1-7.6 |
| Recovery of additions, % | 89 ± 8.9 |
| Recovery after dilution, % | 103 ± 9.2 |

Specificity.

To demonstrate IGFBP-1 C-terminal proteolytic fragment specificity, candidate antibody combinations were assessed for their binding responses to the various IGFBP-1 peptides versus intact (native) IGFBP-1, and to IGFBP-1 in various biological sample pools. As shown in Table 2, the specificity of the IGFBP-1 C-Terminal fragment ELISA was also compared to the specificity of a well established ELISA for Total IGFBP-1 (Diagnostic Systems Laboratories, Inc. Total IGFBP-1 ELISA), which is not affected by variability in the state of phosphorylation of IGFBP-1 or its occupancy by the IGF peptides (24, U.S. Pat. No. 5,747,273). Where the Total IGFBP-1 ELISA demonstrated the expected specificity for intact (native) IGFBP-1 with no cross-reactivity with the C-terminal peptides, the IGFBP-1 C-terminal fragment ELISA demonstrated predominant specificity for the IGFBP-1 C-terminal peptides, particularly the 146-259 fragment, with a low degree of cross-reactivity with the intact (native) IGFBP-1 molecule. Cross-reactivity was calculated as the percentage (%) of the measured level over the expected levels of the potential cross-reactants assayed.

TABLE 2
C-terminal IGFBP-1 Fragment ELISA: Comparative Specificity

| | Assay cross-reactivity, % | |
|---|---|---|
| | Total | C-Terminal |
| Compound | IGFBP-1 | IGFBP-1 |
| Intact IGFBP-1 up to 150 ng/mL | 100 | 10 |
| IGFBP-1 146-259 fragment up to 200 ng/mL | 0 | 100 |
| IGFBP-1 151-259 fragment up to 200 ng/mL | 0 | 15 |
| IGFBP-1 171-259 fragment up to 400 ng/mL | 0 | 0 |

### EXAMPLE 2

### Physiological Investigations

IGFBP-1 C-Terminal Fragment in Physiological Fluids.

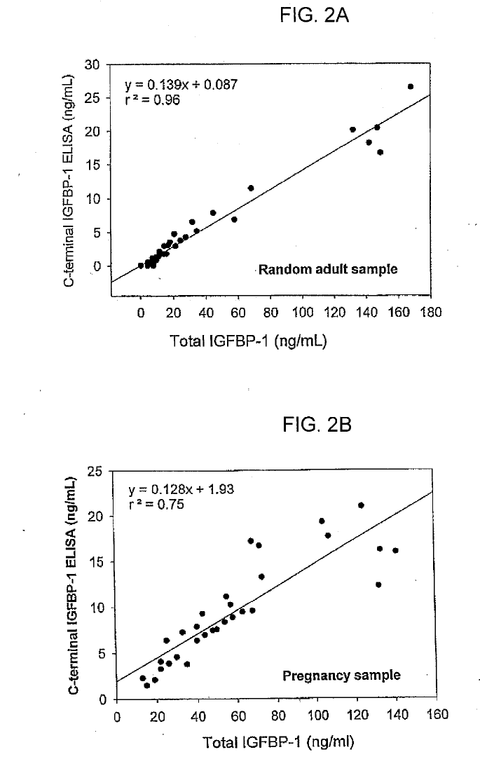

The IGFBP-1 C-terminal proteolytic fragment was measured in random adult serum samples, and in first and second trimester pregnancy sera, using an immunoassay as described herein and the Diagnostic Systems Laboratories, Inc. (Webster, Tex.) Total IGFBP-1 ELISA (FIGS. 2A and 2B).

Random Adult Serum Samples.

In the randomly selected adult serum samples, the IGFBP-1 C-terminal fragment and the total IGFBP-1 were measured using an immunoassay as described herein, and the Diagnostic Systems Laboratories, Inc. (Webster, Tex.) Total IGFBP-1 ELISA (FIG. 2A). The individual values were highly correlated. The C-terminal IGFBP-1 fragment immunoassay detected approximately 10% of the total IGFBP-1 immunoreactivity detected by the DSL Total IGFBP-1 ELISA in the samples. Detection of approximately 10% of the total IGFBP-1 immunoreactivity, as indicated by the slope of the correlation plot in FIG. 2A, is consistent with the demonstrated 10% C-tenninal fragment IGFBP-1 ELISA cross-reactivity with the intact IGFBP-1 molecule (Table 2).

Pregnancy Samples.

In measurements of pregnancy samples, the between-method correlation showed significantly wider scattering of the corresponding data points around the regression line (FIG. 2B) compared to the random adult serum samples, and showed variability between individual samples.

Concentration Ratios.

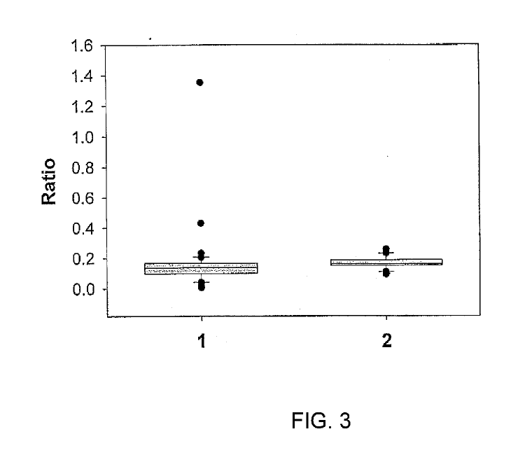

Changes in IGFBP-1 C-terminal proteolytic fragment levels were demonstrated by relating the individual levels of IGFBP-1 C-terminal fragment to the corresponding total IGFBP-1 immunoreactivity. A ratio determination for IGFBP-1 C-terminal fragment and total IGFBP-1 measured in the random adult and pregnancy serum samples described for FIGS. 2A-2B is shown in FIG. 3. The measured ratios (median±95 percentile limits) show a tight distribution for random adult serum samples, and significantly higher variability in the pregnancy samples.

The following references are cited herein:

1. Rotwein P. Structure, evolution, expression and regulation of insulin-like growth factors I and II [Review]. Growth Factors 1991; 5:3-18.
2. Daughaday W H, Rotwein P. Insulin-like growth factors I and II. Peptide, messenger ribonucleic acid and gene structures, serum and tissue concentration. Endocr Rev 1989; 10:68-91.
3. LeRoith D, Adamo M, Werner H, Roberts C T, Jr. Insulin-like growth factors and their receptors as growth regulators in normal physiology and pathological states [Review]. Trends Endocrinol Metab 1991; 2:134-139.
4. Jones J I, Clemmons D R. Insulin-like growth factors and their binding proteins: biological actions. Endocr Rev 1995; 16:3-34.
5. Cohen P, Rosenfeld R G. Physiologic and clinical relevance of the insulin-like growth factor binding proteins [Review]. Curr Opin Pediatr 1994; 6:462-467.
6. Rajarm S, Baylink D J, Mohan S. Insulin-like growth factor-binding proteins in serum and other biological fluids: regulation and functions. Endocrine Rev 1997; 18:801-831.
7. Koistinen R, Kalkkinen N, Huhtala M L, Seppala M, Bohn H, Rutanen E-M. Placental protein 12 is a decidual protein that binds somatomedin and has an identical N-terminal amino acid sequence with somatomedin binding protein from human amniotic fluid. Endocrinology 1986; 118:1375-1378.
8. Bell S C, Keyte J W. N-terminal amino acid sequence of human pregnancy-associated endometrial alpha 1-globulin, an endometrial insulin-like growth factor (IGF) binding protein: evidence for two small molecular weight IGF binding proteins. Endocrinology 1988; 123:1202-1204.
9. Lee P D K, Conover C A, Powell D R. Regulation and function of insulin-like growth factor binding protein-1. Proc Soc Exp Biol Med 1993; 204:4-29.
10. Suikkari A-M, Jalkanen J, Koistinen R, Butzow R, Ritvos 0, Ranta T, Seppala M. Human granulosa cells synthesize low molecular weight insulin-like growth factor-binding protein. Endocrinology 1989; 124:1088-1090.
11. Giudice L C. Insulin-like growth factors and ovarian follicular development. Endocr Rev 1992; 13:641-669.
12. Rutanen E-M, Bohn H, Seppala M. Radioimmunoassay of placental protein 12: levels in amniotic fluid, cord blood, and serum of healthy adults, pregnant women, and patients with trophoblastic disease. Am J Obstet Gynecol 1982; 144:460-463.
13. Hills F A, English J, Chard T. Circulating levels of IGF-I and IGF-binding protein-1 throughout pregnancy: relation to birth weight and maternal weight. J Endocrinol 1996; 148:303-309.
14. Verhaeghe J, Billen J, Giudice L C. Insulin-like growth factor-binding protein-1 in umbilical artery and vein of term fetuses with signs suggestive of distress during labor. J Endocrinol 2001; 170: 585-590.
15. Kajantic E. Insulin-like growth factor (IGF)-I, IGF binding protein (IGFBP)-3, phosphoisoforms of IGFBP-1 and postnatal growth in very-low-birth-weight infants. Horm Res 2003; 60: 124-130.
16. Sakai K, D'Ercole A J, Murphy L J, Clemmons D R. Physiological differences in insulin-like growth factor binding protein-1 phosphorylation in IGFBP-1 transgenic mice. Diabetes 2001; 50:32-38.
17. Watson C, Bialek P, Khosravi J, Yee Siu-Pok, Han V. Elevated circulating insulin-like growth factor binding protein-1 is sufficient to cause fetal growth restriction. 2005 (in preparation).
18. Jones J I, D'Ercole A J, Camacho-Hubner C, Clemmons D R. Phosphorylation of insulin-like growth factor (IGF)-binding protein 1 in cell culture and in vivo: effects on affinity for IGF-I. Proc Natl Acad Sci USA 1991; 88:7481-7485.
19. Koistinen R, Angervo M, Leinonen P, Hakala T, Seppala M. Phosphorylation of insulin-like growth factor-binding protein-1 increases in human amniotic fluid and decidua from early to late pregnancy. Clin Chem Acta 1993; 215:189-199.
20. Westwood M, Gibson J M, Davies A J, Young R J, White A. The phosphorylation pattern of insulin-like growth factor-binding protein-1 in normal plasma is different from that in amniotic fluid and changes during pregnancy. J Clin Endocrinol Metab 1994; 79:1735-1741.
21. Westwood M, Gibson J M, Williams A C, Clayton P E, Hamberg O, Flyvbjerg A, White A. Hormonal regulation of circulating IGFBP-1 phosphorylation Status. J Clin Endocrinol Metab 1995; 80:3520-3527.
22. Bankowski E, Sobolewski K, Palka J, Jawoski S. Decreased expression of the insulin-like growth factor-I-binding protein-1 (IGFBP-1) phosphoisoform in pre-eclampsic Warton's jelly and its role in the regulation of collagen biosynthesis. Clin Chem Lab Med 2004; 42:175-181.
23. Heald A H, Siddals K W, Fraser W, Taylor W, Kaushal K, Morris J, Young R J, White A, Gibson J M. Low circulating levels of insulin-like growth factor binding protein-1 (IGFBP-1) are closely associated with the presence of marcrovascular disease and hypertension in type 2 diabetes. Diabetes 2002; 18:2629-2636.
24. Khosravi M J, Diamandi A, Mistry J. Immunoassay of insulin-like growth factor binding protein-I. Clin Chem 1997; 43:523-532.
25. Rechler M M. Editorial: Growth inhibition by insulin-like growth factor (IGF) binding protein-3—What's IGF got to do with it. Endocrinology 1997; 138:2645-2647.
26. Giudice L C. Editorial: IGF binding protein-3 protease regulation: How sweet it is. J Clin Endocrinol Metab 1995; 80:2279-2281.
27. Rajah R, Katz L, Nunn S, Solberg P, Beers T, Cohen P. Insulin-like growth factor binding protein (IGFBP) proteases: Functional regulations of cell growth. Prog Growth Factor Res1995; 6:273-284.
28. Hossenlopp P, Sergovia B, Lassarre C, Roghani M, Bredon M, Binoux M. Evidence of enzymatic degradation of insulin-like growth factor binding proteins (IGFBPs) in the 150 K complex during pregnancy. J Clin Endocrinol Metab 1990; 71:797-805.
29. Lawrence J B, Oxvig C, Overgaard M T, Sottrup-Jensen L, Gleich G J, Hays L G, Yates J R, Conover C. The insulin-like growth factor (IGF)-dependent IGF binding protein-4 protease secreted by human fibroblasts is pregnancy-associated plasma protein-A. Proc Natl Acad Sci USA 1999; 96:3149-3153.
30. Lassarre C, Duron F, Binoux M. Use of ligand immunofunctional assay for human insulin-like growth factor (IGF) binding protein-3 (IGFBP-3) to analyze IGFBP-3 proteolysis and IGF-I bioavailability in healthy adults, GH-deficient and acromegalic patients, and diabetics. J Clin Endocrinol Metab 2001; 86: 1942-1952.
31. Diamandi A, Mistry J, Krishna R G, Khosravi J ((SRA). Immunoassay of insulin-like growth factor binding protein-3: new means to quantifying IGFBP-3 proteolysis. J Clin Endocrinol Metab 85; 2327-2333, 2000.
32. Gravett M G, Novy M J, Rosenfeld R G, Reddy, A P, Jacob T, et al., Diagnosis of intra-amniotic fluid infection by proteomic and identification of novel biomarkers., JAMA 2004; 292:462-469
33. Sala A, Capaldi S, Campagnoli M, Faggion B, Labo S, et al. Structure and properties of the C-terminal domain of insulin-like growth factor protein-I isolated from human amniotic fluid. J Biol Chem 2005; 280: 29812-29819
34. Schebek-Furstenberg V, Standker L, Oppermann M, Muller-Wiefel D E, Hahn W, et al. IGF-binding protein-3 fragments in plasma of a child with acute renal failure. Pediatr Nephrol 19:1418-25
35. Powell D R, Lee P D K, Shively J E, Eckenhausen M, Hintz R L. Method for purification of an insulin-like growth factor-binding protein produced by human HEP-G2 hepatoma cells. J Chromatogr B Biomed Appl 1987; 420:163-170
36. Khosravi M J, Morton R C. Novel application of streptavidin-hapten derivative as protein-tracer conjugate in competitive-type immunoassays involving biotinylated detection probes. Clin Chem 1991; 37:58-63.
37. Khosravi M J, Papanastasiou-Diamandi A, Mistry J. Ultrasensitive immunoassay for prostate-specific antigen based on conventional colorimetric detection. Clin Biochem 1995; 28:407-414.
38. Khosravi M J, Diamandis E P. Immunofluorometry of choriogonadotropin by time-resolved fluorescence spectroscopy, with a new europium chelate as label. Clin Chem 1987; 33:1994-1999.
39. LeRoith D, Roberts C T Jr. The insulin-like growth factor system and cancer. Cancer Lett 2003; 195:127-37.
40. Cohen P. Serum insulin-like growth factor-I levels and prostate cancer risk—interpreting the evidence. J Natl Cancer Inst 1998; 90:876-879.
41. Chan J M, Stampfer M J, Giovannucci E, Gann P H, Ma J, Wilkinson P, Hennekens C H, Pollak M. Plasma insulin-like growth factor-I and prostate cancer risk: A prospective study. Science 1998; 279:563-566.
42. Khosravi J, Diamandi A, Mistry J, Scorilas A. Insulin-like growth factor I (IGF-1) and IGF-Binding protein-3 in benign prostatic hyperplasia and prostate cancer. J Clin Endocrinol Metab 2001; 86: 694-699.
43. Rosen C J, Pollak M. Circulating IGF-I: new perspectives for a new century. TEM 1999; 10:136-141
44. Fields, G. B., and Noble, R. L., Solid phase peptide synthesis utilizing fluorenylmethoxycarbonyl amino acids. Int. J. Peptide, Proteins Res. (1990), 35: 161-214.
45. Chicz, R. M., and Regnier, F. E ., High pressure liquid chromatography: Effective protein purification by various chromatographic modes. Meth. Enzymol., 1990, 82: 392-421.
46. Shaw, C., Peptide purification by reverse phase HPLC. In: basic protein and peptide protocols, Methods in Mol. Biol. Series., Walker, J. M (ed), Humans Press, NJ, 1994, 32: 275-288.
47. Harlow, E., and Lane, D., Antibodies: A laboratory manual, Cold Spring Harbor, N.Y., 1988.
48. Hwa, V., Oh, y., and Rosenfeld, R. G., The insulin-like growth factor-binding protein (IGFBP) superfamily, Endocr Rev. 1999: 20: 761-87.
49. Sambrook, J., and Russell, D., Molecular cloning: A laboratory manual, Cold Spring Harbor, N.Y., 2001.
50. Graves J D, Krebs E G. Protein phosphorylation and signal transduction. Pharmacol Ther 1999; 82: 111-121.
51. Rott I. The primary interaction with antibody in “essential immunology” Blackwell Scientific Publications, Boston Mass., 1994; 8^{th} edition: 81-102.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. An immunoassay system comprising a first antibody and a second antibody, wherein the first antibody binds to a proteolytic epitope of a protein fragment, and wherein the second antibody binds to the protein fragment.

2. The immunoassay system of claim 1, wherein the protein fragment is an IGFBP fragment.

3. The immunoassay system of claim 2, wherein the IGFBP fragment is selected from the group consisting of an IGFBP-1 fragment, an IGFBP-3 fragment, and an IGFBP-5 fragment.

4. The immunoassay system of claim 1, wherein the proteolytic epitope comprises a portion of an amino acid sequence selected from the group consisting of SEQ ID NO. 3, SEQ ID NO. 4, and SEQ ID NO. 5.

5. The immunoassay system of claim 1, wherein the protein fragment comprises SEQ ID NO. 3.

6. The immunoassay system of claim 1, further comprising a solid support coupled with the first antibody.

7. The immunoassay system of claim 6, wherein the solid support comprises a protein coupling surface selected from the group consisting of a microtiter plate, a colloidal metal particle, and iron oxide particle and a polymeric bead.

8. The immunoassay system of claim 1, wherein the second antibody is coupled with a label.

9. The immunoassay system of claim 8, wherein the label comprises a chemiluminescent agent, a colorimetric agent, an energy transfer agent, an enzyme, a substrate of an enzyme reaction, a fluorescent agent or a radioisotope.

10. The immunoassay system of claim 9, wherein the enzyme is selected from the group consisting of alkaline phosphatase, amylase, luciferase, catalase, beta-galactosidase, glucose oxidase, glucose-6-phosphate dehydrogenase, hexokinase, horseradish peroxidase, lactamase, urease, and malate dehydrogenase.

11. An immunoassay method for measuring an amount of a protein fragment in a sample, comprising the steps of:
(a) binding a first antibody to a proteolytic epitope of a protein fragment in a sample, thereby creating a bound first antibody;
(b) binding a second antibody to the protein fragment, thereby creating a bound second antibody;
(c) measuring an amount of the bound second antibody; and
(d) measuring an amount of the protein fragment in the sample based on the amount of the bound second antibody.

12. The immunoassay method of claim 11, wherein the protein fragment is an IGFBP fragment.

13. The immunoassay method of claim 12, wherein the IGFBP is selected from the group consisting of IGFBP-1, IGFBP-3, and IGFBP-5.

14. The immunoassay method of claim 11, wherein the proteolytic epitope comprises a portion of an amino acid sequence selected from the group consisting of SEQ ID NO.3, SEQ ID NO.4, and SEQ ID NO.5.

15. The immunoassay method of claim 11, wherein the protein fragment comprises SEQ ID NO.3.

16. The immunoassay method of claim 11, further comprising a solid support coupled with the first antibody.

17. The immunoassay method of claim 16, wherein the solid support comprises a protein coupling surface selected from the group consisting of a microtiter plate, a colloidal metal particle, and iron oxide particle and a polymeric bead.

18. The immunoassay method of claim 11, wherein the second antibody is coupled with a label.

19. The immunoassay method of claim 18, wherein the label comprises a chemiluminescent agent, a colorimetric agent, an energy transfer agent, an enzyme, a substrate of an enzyme reaction, a fluorescent agent or a radioisotope.

20. The immunoassay method of claim 19, wherein the enzyme is selected is selected from the group consisting of alkaline phosphatase, amylase, luciferase, catalase, beta-galactosidase, glucose oxidase, glucose-6-phosphate dehydrogenase, hexokinase, horseradish peroxidase, lactamase, urease, and malate dehydrogenase.

21. The method of claim 11, wherein the sample comprises a biological fluid.

22. The method of claim 21, wherein the biological fluid is selected from the group consisting of non-pregnant serum, pregnancy serum, and amniotic fluid.

23. The immunoassay method of claim 12, further comprising the steps of:
(e) measuring an amount of total IGFBP in the sample, and
(f) relating the amount of the IGFBP fragment to the amount of total IGFBP in the sample.

24. The immunoassay method of claim 23, wherein the IGFBP is selected from the group consisting of IGFBP-1, IGFBP-3, and IGFBP-5.

25. The immunoassay method of claim 23, wherein relating the amount of the IGFBP fragment to the amount of total IGFBP in the sample comprises calculating a ratio of the amount of the IGFBP fragment and the amount of total IGFBP in the sample.

26. An immunoassay kit for measuring an amount of a protein fragment in a sample, comprising:
(a) a first antibody and a second antibody, wherein the first antibody binds to a proteolytic epitope in a protein fragment and the second antibody binds to the protein fragment;
(b) a solid support coupled with the first antibody; and
(c) a label coupled with the second antibody.

27. The immunoassay kit of claim 21, wherein the protein fragment is an IGFBP fragment.

28. The immunoassay kit of claim 22, wherein the IGFBP fragment is selected from the group consisting of an IGFBP-1 fragment, an IGFBP-3 fragment, and an IGFBP-5 fragment.
